(19) United States
(12) Patent Application Publication
(10) Pub. No.: US 2007/0092442 A1
(43) Pub. Date: Apr. 26, 2007

(54) F-18-fluorinated phosphonium cation imaging agents and methods of synthesis

(76) Inventors: Sanjiv S. Gambhir, Portolla Valley, CA (US); Zhen Cheng, Mountain View, CA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/584,936

(22) Filed: Oct. 23, 2006

Related U.S. Application Data
(60) Provisional application No. 60/729,255, filed on Oct. 21, 2005.
(60) Provisional application No. 60/830,563, filed on Jul. 13, 2006.

(51) Int. Cl. A61K 51/00

(52) U.S. Cl. 424/1.11

(57) ABSTRACT

The present disclosure provides mitochondrial imaging probes, particularly imaging probes capable of imaging changes in mitochondrial membrane potential and conditions associated with mitochondrial dysfunction.

### CROSS-REFERENCE TO RELATED APPLICATIONS

This application claims priority to copending U.S. provisional patent applications Ser. No. 60/729,255, entitled “MATRIX ASSISTED LASER DESORPTION IONIZATION (MALDI) SUPPORT STRUCTURES AND METHODS OF MAKING MALDI SUPPORT STRUCTURES” filed on Oct. 21, 2005, and Ser. No. 60/830,563, entitled “F-18-FLORINATED PHOSPHONIUM CATION IMAGING AGENTS AND METHODS OF SYNTHESIS” filed on Jul. 13, 2006, both of which are entirely incorporated herein by reference.

### FIELD OF THE INVENTION(S)

The present disclosure relates to the field of imaging agents, particularly to imaging probes capable of imaging mitochondria, even more particularly to imaging probes capable of imaging changes in mitochondrial membrane potential, and conditions associated with mitochondrial dysfunction.

### BACKGROUND

A wide range of diseases, including cancers, diabetes, heart failure, cardiovascular and liver diseases, AIDS, degenerative diseases, autoimmune disorders, and the pathophysiology of aging are associated with mitochondria dysfunction, and mitochondrial dysfunction is increasingly implicated in other conditions and disorders. It has recently become widely accepted that mitochondria play a key role during apoptosis. Dramatic changes of mitochondrial membrane potential (Δψₘ) are associated with these mitochondrial diseases. For instance, the difference in Δψm between normal epithelial cells and many carcinoma cells is at least 60 mV, and the loss of Δψₘ is an early event in cell apoptosis caused by pro-apoptotic agents.

Alteration in Δψₘ is an important characteristic of a vast array of pathologies that either involve suppressed (e.g., cancer) or enhanced apoptosis (e.g., HIV, degenerative disease) as well as >100 diseases directly caused by mitochondrial dysfunction such as DNA mutations and oxidative stress (e.g., various types of myopathies).

Lipophilic cations, such as the rhodamine-123 (Rh123) and tetraphenylphosphonium (TPP) salts, can penetrate the plasma and mitochondrial membranes and selectively accumulate in mitochondria, because of the negative inner mitochondrial transmembrane potential (−120 to −170 mV, negative inside).

There are technetium complexes, derivatives of [⁹⁹mTc]annexin V, for apoptosis imaging by using SPECT. However, these currently available technetium labeled mitochondria imaging agents are hampered by several limitations. More particularly, labeling a molecule with ⁹⁹mTc requires a conjugating moiety to complex the technetium ion such that Tc-based imaging agents have a high molecular weight which reduces the permeability of the imaging agent in target areas. Further, technetium imaging agents are imaged with SPECT, which has relatively low spatial resolution and sensitivity when compared to comparable PET images. Also, such annexin V derivatives provide images due to overexpression of specific membrane proteins, which is only detectable for a limited amount of time.

The collapse of Δψₘ is considered the point of no return of the apoptotic process. Therefore, the collapse of Δψₘ affords the earliest time point to detect apoptosis, rather the last event as in the case of annexin V, and the collapse persists independent of time. It would be desirable to have an imaging agent/probe that has an affinity for mitochondria, and that whose uptake is related to Δψₘ.

Additionally, current approaches for evaluation of efficacy of chemotherapy rely on alterations in tumor growth rate, a costly approach of limited sensitivity that involves months of follow up, repeated visits in clinic, multiple radiographic scans and frequently a number of treatment cycles. In view of the high incidence of cancer cases (about 1.3 million per year in the United States), the high frequency of chemotherapy applications and the low frequency of successful chemotherapy, there is an urgent need for a non-invasive imaging probe of rapid and sensitive assessment of tumor response to treatment.

There also exists a great need to diagnose and image cardiovascular diseases and disorders, many of which are associated with mitochondrial dysfunction. Thus, there is also an urgent need for non-invasive imaging probes for rapid and sensitive measurement of cardiac uptake of imaging agents having an affinity for dysfunctional mitochondria for the imaging of cardiovascular diseases such as myocardial perfusion.

### SUMMARY

Embodiments of the present disclosure include an imaging probe of the formula (4-[¹⁸F]fluorophenyl)triphenylphosphonium (¹⁸FTPP). The imaging probes of the present disclsoure are taken up by mitochondria, where the uptake is related to changes in mitochondiral membrane potential (Δψₘ). The present disclosure also includes imaging compounds including ¹⁸FTPP. Pharmaceutically acceptable imaging compositions according to the present disclosure include one or more imaging probes according to the present disclosure (e.g., ¹⁸FTPP), or a pharmaceutically acceptable salt thereof, combined with a pharmaceutically acceptable carrier and/or excipient. In some embodiments, a pharmaceutically acceptable salt includes an ¹⁸FTPP cation and a pharmaceutically acceptable anion. In some embodiments the anion is selected from I⁻, Cl⁻, and Br⁻.

The present disclosure also includes methods of imaging changes in mitochondiral membrane potential (Δψₘ), mitochondrial dysfunction, and conditions and/or diseases associated with such dysfunction. Such methods include administering a detectably effective amount of ¹⁸FTPP or a pharmacutically acceptable salt thereof, or a pharmacuetically acceptable composition thereof to a host; using an imaging apparatus to create a radiographic image and image the location and distribution of the imaging agent in the host. The imaging apparatus used to detect and monitor the imaging agent include imaging technologies suitable for the particular label, for example, a gamma camera, a PET apparatus, a SPECT apparatus. In an exemplary embodiment, the imaging apparatus is a PET apparatus.

The above methods can also be used to detect a change in mitochondiral membrane potential. Furthermore the imaging methods can be used for diagnosing and/or monitoring diseases associated with a change in mitochondrial membrane potential. In some such methods, the disease and/or condition diagnosed and/or monitored is selected from cancer, diabetes, heart failure, cardiovascular diseases, liver diseases, AIDS, degenerative diseases, autoimmune disorders, myopathies, and conditions associated with aging.

The present disclosure also includes methods of determining the effectiveness of a drug on various conditions associated with increased or decreased apoptosis. Such methods include administering an amount of the drug to a host; administering a detectably effective amount of a composition comprising (4-[¹⁸F]fluorophenyl)triphenylphosphonium (¹⁸FTPP) or a pharmacutically acceptable salt thereof to a host; creating a radiographic image of the location and distribution of the ¹⁸FTPP in the host with an imaging apparatus; and determining an amount of ¹⁸FTPP taken up by host mitochondria, wherein the amount of uptake by host mitochondria is related to the effect of the drug on apoptosis in host cells.

The present disclosure also includes novel methods of synthesizing ¹⁸FTPP. One embodiment of a method of synthesizing ¹⁸FTPP includes a two-step process including direct coupling of triphenylphosphine (PPh3) with 4-[¹⁸F]Fluoroidodobenzene ([¹⁸F]FIB). In an embodiment, the two step process includes the following steps: (1) synthesis of no-carrier added [¹⁸F]FIB using 1-trimethylamino-4-iodobenzene as a precursor; and (2) directly coupling the [¹⁸F]FIB produced in step 1 with PPh₃ to form ¹⁸FTPP. In another embodiment, a method of synthesizing ¹⁸FTPP includes a one-step process including direct nucleophilic substitution of no-carrier-added [¹⁸F]fluoride with the precursor 4-nitrophenyltriphenylphosphonium.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### BRIEF DESCRIPTION OF THE DRAWINGS

Aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates the chemical structure, and molecular mass (M.W.) of a library of phosphonium cations: (4-bromobutyl) triphenyl-phosphine bromide (Compound #1, BrTPP), butyltriphenylphosphonium chloride (Compound #2, BuTPP), (4-carboxybutyl)triphenyl-phosphonium bromide (Compound #3, CoTPP), methyltriphenyl phosphonium bromide (Compound #4, MeTPP), tetraphenylphosphonium bromide (Compound #5, TPP), triphenyl(2-pyridylmethyl) phosphonium chloride hydrochloride (Compound #6, PyTPP), tetrabutylphosphonium bromide (Compound #7, TBuP), 4-Fluorophenyltriphenyl phosphonium (Compound #8, FTPP), tetra-4-fluorophenylphosphonium iodide (Compound #9, F4TPP), and 4-fluorobenzyl-triphenylphosphonium iodide (Compound #10, FBnTP).

FIG. 2 illustrates C6 cell uptake of various phosphonium cations over time at room temperature as determined by MALDI-TOF-MS. Values are expressed as mean percentage of cell uptake ±S.D. of three independent determinations.

FIG. 3 illustrates the effects of various concentrations of protonophore CCCP, K⁺-ionophore valinomycin, and high K⁺HEPES buffer on the uptake of 5 μM FTPP. Each value represents the mean of three independent experiments. Values are expressed as mean percentage of normalized uptake ±S.D. of three independent experiments.

FIG. 4 illustrates one embodiment of a method of synthesis for 4-[¹⁸F]Fluorophenyltriphenylphosphonium cation.

FIG. 5 illustrates another embodiment of a method of synthesis for 4-[¹⁸F]Fluorophenyltriphenylphosphonium cation.

FIG. 6A is a scanned image of decay-corrected whole-body coronal and transverse microPET images of a normal nude mouse at 0.5, 1, 2, and 4 hr (10-min static image) after injection of 15 μCi of [¹⁸F]FTPP. FIG. 6B illustrates time-activity curves of [¹⁸F]FTPP for liver, heart, and muscle derived from a multiple time-point microPET study. ROIs are shown as the mean % ID/g ±SD (n=3).

### DETAILED DESCRIPTION

Embodiments of the present disclosure will employ, unless otherwise indicated, techniques of synthetic organic chemistry, biochemistry, molecular biology, and the like, that are within the skill of the art. Such techniques are explained fully in the literature.

The following examples are put forth so as to provide those of ordinary skill in the art with a complete disclosure and description of how to perform the methods and use the compositions disclosed and claimed herein. Efforts have been made to ensure accuracy with respect to numbers (e.g., amounts, temperature, etc.) but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible. All patents, patent applications, and publications mentioned herein are hereby incorporated by reference in their entireties.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. It must be noted that, as used in the specification and the appended claims, the singular forms “a,” “an” and “the” include plural referents unless the context clearly dictates otherwise. Thus, for example, reference to “a support” includes a plurality of supports.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range.

In this specification and in the claims that follow, reference will be made to a number of terms that shall be defined to have the following meanings unless a contrary intention is apparent.

Definitions:

As used herein, the term “imaging probe”, “imaging agent”, or imaging compound refers to the radiolabeled compounds of the present disclosure that are capable of serving as mitochondrial imaging agents and whose uptake is related to Δψₘ, such as the radiolabeled phosphonium cations of the present disclosure (e.g., ¹⁸FTPP).

A “pharmaceutically acceptable carrier” refers to a biocompatible solution, having due regard to sterility, pH, isotonicity, stability, and the like and can include any and all solvents, diluents (including sterile saline, Sodium Chloride Injection, Ringer's Injection, Dextrose Injection, Dextrose and Sodium Chloride Injection, Lactated Ringer's Injection and other aqueous buffer solutions), dispersion media, coatings, antibacterial and antifungal agents, isotonic agents, and the like. The pharmaceutically acceptable carrier may also contain stabilizers, preservatives, antioxidants, or other additives, which are well known to one of skill in the art, or other vehicle as known in the art.

As used herein, “pharmaceutically acceptable salts” refer to derivatives of the disclosed compounds wherein the parent compound is modified by making non-toxic acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, mesylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

The pharmaceutically acceptable salts of the present disclosure can be synthesized from a parent compound that contains a basic or acidic moiety by conventional chemical methods. Generally, such salts can be prepared by reacting free acid forms of these compounds with a stoichiometric amount of the appropriate base (e.g., Na, Ca, Mg, or K, hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts may be found, e.g., in Remington's Pharmaceutical Sciences, 17th ed., Mack Publishing Company, Easton, Pa., p. 14¹⁸F (1985), which is hereby incorporated by reference in relevant part.

By “administration” is meant introducing a compound into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used.

As used herein, the term “host”, “organism”, “individual” or “subject” includes humans, mammals (e.g., cats, dogs, horses, etc.), living cells, and other living organisms. A living organism can be as simple as, for example, a single eukaryotic cell or as complex as a mammal. “Patient” refers to an individual or subject who has undergone, is undergoing, or will undergo treatment.

Discussion

The development of radiolabeled mitochondriotropic molecular probes may provide a powerful tool to investigate the role of mitochondria in the pathophysiology and treatment of various disorders associated with mitochondria dysfunction using a non-invasive imaging technology such as positron emission tomography (PET). Such probes would be useful for the analysis and treatment of disorders such as cancers, diabetes, heart failure, cardiovascular and liver diseases, AIDS, degenerative diseases, autoimmune disorders, the pathophysiology of aging, and other conditions and disorders associated with and/or characterized by mitochondrial dysfunction. Furthermore, drugs based on the changes in Δψₘ may serve as a novel, effective approach for disease treatment.

Many phosphonium and other lipophilic cations have been evaluated as anticancer drugs, as well as carriers to deliver passenger molecules selectively to the mitochondria of cancer cells, for manipulation of these cells. ³H-labeled phosphonium analogs can function as a molecular probe for selective accumulation in certain cancer cells. Several radiolabeled phosphonium cations, [¹¹C]methyltriphenylphosphonium, ²²3-[¹⁸F]Fluoropropyl and 4-[¹⁸F]Fluorobenzyltriarylphosphonium, have also been synthesized and evaluated as mitochondrial targeting agents.

As demonstrated in example 1 below and in copending provisional application 60/729,255, which is incorporated herein by reference, the mitochondria targeting ability of a library of phosphonium analogs was studied using matrix-assisted laser desorption/ionization time of flight mass spectrometry (MALDI-TOF-MS), and it was found that (4-[¹⁹F]fluorophenyl)-triphenylphosphonium (¹⁹FTPP or FTPP) exhibited 80% of the TPP uptake in glioma C6 cells in vitro, which is much higher accumulation than that of methyltriphenylphosphonium and butyltriphenylphosphonium. Cell uptake studies, described in greater detail in the examples below, also demonstrated that the uptake of FTPP in C6 tumor cells is dependent on the mitochondrial membrane potential of the cells.

The present disclosure provides novel non-invasive imaging probes and imaging compounds including such probes. Embodiments of the present disclosure also include pharmaceutical compositions including imaging probes of the present disclosure and at least one pharmaceutically acceptable carrier or excipient. The imaging probes and imaging compositions of the present disclosure exhibit high affinity for mitochondria, are sensitive to changes in Δψₘ, and can aid in detection of dysfunctional mitochondria having abnormal (e.g., enhanced or suppressed) activity. In an exemplary embodiment the imaging probe includes (4-[¹⁸F]fluorophenyl)triphenylphosphonium (¹⁸FTTP).

The imaging probes of the present disclosure are suitable for use in imaging or assessment, particularly PET or SPECT imaging, of mitochondrial dysfunction in a patient. Some exemplary embodiments of non-radioactive elements and their counterparts that can be used in the imaging probes of the present disclosure include, but are not limited to, F-19 (F-18), C-12 (C-11), I-127 (I-125, I-124, I-131, I-123), CI-36 (CI-32, CI-33, CI-34), Br-80 (Br-74, Br-75, Br-76, Br-77, Br-78), Re-185/187 (Re-186, Re-188), Y-89 (Y-90, Y-86), Lu-177, and Sm-153. Preferred probes of the present disclosure are labeled with one or more radioisotopes, preferably including ¹¹C, ¹⁸F, ⁷⁶Br, ¹²³I, ¹²⁴I, or ¹³¹I and more preferably ¹⁸F, ⁷⁶Br, or ¹²³I, ¹²⁴I or ¹³¹I and are suitable for use in peripheral medical facilities and PET clinics.

The present disclosure provides imaging probes of the formula ¹⁸FTTP that are taken up by mitochondria and whose uptake is proportional to Δψₘ. This allows detection and imaging of dysfunctional mitochondria, e.g., mitochondria with suppressed or enhanced activity. In embodiments, the present disclosure provides imaging agents including a radiolabeled labeled lipophilic cation, particularly a lipophilic phosphonium salt of the present disclosure (e.g., ¹⁸FTPP) having one or more radioisotopes and capable of associating with (e.g., binding to, or being taken up by) dysfunctional mitochondria. More particularly, the imaging probes of the present disclosure are suitable for use in measuring mitochondrial membrane potential (Δψₘ) in vivo under a variety of conditions where the radiation emitted by the radioisotope of the probe is utilized to form the image. In preferred embodiments, radiolabeled imaging probes of the present disclosure comprise one or more radioisotopes capable of emitting positron radiation and are suitable for use in positron emission tomography (PET) (e.g., ¹⁸F). In a particularly preferred embodiment the imaging probe is ¹⁸FTTP.

According to yet another aspect, the present disclosure provides pharmaceutical compositions comprising imaging probes, particularly ¹⁸FTPP, or the pharmaceutically acceptable salts or solvates thereof, which compositions are useful for imaging variations in mitochondrial surface potential (Δψₘ), imaging cells or tissues having dysfunctional mitochondria, and imaging and/or monitoring diseases or conditions associated with dysfunctional mitochondria (e.g., various cancers, diabetes, heart failure, cardiovascular and liver diseases, AIDS, degenerative diseases, autoimmune disorders, aging, and other myopathies). Pharmaceutically acceptable salts of the imaging compounds of the present disclosure include a radiolabeled cation, such as ¹⁸FTPP, and a pharmaceutically acceptable anion. Suitable anions include, but are not limited to, Br⁻, Cl⁻, and I⁻. The present disclosure further provides methods of imaging patients suffering from any of the above-recited diseases or disorders with an effective amount of a salt or composition of the present disclosure.

The imaging compounds of the present disclosure, particularly ¹⁸FTTP and/or salts thereof, have a distribution profile in the body that is a function of mitochondrial integrity and are suitable for use as diagnostic tools in the identification and imaging of various diseases and disorders associated with mitochondrial dysfunction. Moreover, the compounds of the present disclosure are useful diagnostic tools for assessing the efficacy of existing therapeutic drugs as well as the development of novel drugs. For example, the effectiveness of drugs that trigger apoptosis (e.g., anticancer drugs) or suppress apoptosis (e.g., drugs that block the degenerative process in HIV) can be assessed by determining if the administration of said drugs produces a change in Δψₘ. The effect can be monitored by observing the change to Δψₘ by measuring Δψₘ using the compounds and imaging methods of the present disclosure.

The present disclosure also includes methods of targeting drug delivery to the mitochondria for the treatment of various conditions and disorders associated with mitochondrial dysfunction, including various conditions associated with increased or decreased apoptosis. FTTP can be used as a carrier for drug delivery for non-imaging applications, while ¹⁸FTPP can be used as a carrier for drug delivery applications where it is desirable to image the delivery of the drug to the target, such as to confirm delivery of the drug to the designated target, to determine the biodistribution, bioavailability, and elimination of a drug, and the like. In embodiments, the FTTP or ¹⁸FTPP is coupled to a drug for targeted delivery to the mitochondria.

Embodiments of the present disclosure further provide methods of imaging including the steps of: providing at least one radiolabeled imaging compound of the disclosure or a salt thereof, where the imaging compound includes a pharmaceutically acceptable anion and at least one radiolabeled cation according to the present disclosure; contacting cells or tissues with the radiolabeled salt; and making a radiographic image.

The imaging methods provided by the present disclosure are suitable for assessing mitochondrial membrane potential (Δψₘ). More particularly, the imaging methods of the present disclosure are suitable for measuring changes in mitochondrial membrane potential over time to assess the efficacy of therapeutic protocols or pharmaceutical treatments. Cells that exhibit suppressed or enhanced rates of apoptosis frequently also exhibit decreased or increased mitochondria activity. The imaging probes/compounds provided by the present disclosure typically localize to cells in a concentration proportional to the level of mitochondria activity. Thus, frequently when cells are experiencing reduced levels of apoptosis (e.g., cancer cells), a greater portion of the imaging compound of the present disclosure (e.g. ¹⁸FTPP) administered to the patient localizes to those cells and vice versa; cells with enhanced levels of apoptosis (e.g., auto immune disorders, tumor cells responsive to chemotherapy agents) will accumulate less ¹⁸FTPP than normal cells. Thus, the imaging methods of the present disclosure are suitable for use in the imaging of cells, tissues, or other physiological targets that are experiencing suppressed or enhanced apoptosis.

The imaging methods of the present disclosure are generally suitable for imaging any disease, disorder, or pathology related to mitochondria. Preferred diseases and disorders that are suitable for imaging by the methods and compounds of the present disclosure include, but are not limited to, cancer (including neoplasms), cardiovascular diseases (including infraction and perfusion), liver diseases, degenerative diseases or disorders, autoimmune disorders, aging, HIV infections, myopathies caused by oxidative stress or DNA mutation, or other diseases and disorders associated with mitochardial dysfunction.

The imaging methods of the present disclosure are also suitable for use in assessing the efficacy of therapeutic drugs capable of triggering or suppressing apoptosis. The imaging methods of the present disclosure may also be used to assess the efficacy of chemotherapy or radiation treatment protocols used to retard or destroy cancer and other malignant tumors. The imaging methods of the present disclosure are also suitable in developing new therapeutic agents capable of disrupting mitochondrial function in target tissue.

The radiolabeled imaging compounds of the present disclosure including ¹⁸FTPP and imaging methods using these compounds provide a non-invasive approach for early and sensitive assessment of treatment efficacy within a few days of starting a therapeutic protocol, as compared to current assessment methods, which may require months. Most major anticancer drugs (e.g., taxol, cisplatin, vinblastine, and etoposide) induce their apoptotic effect via a cascade of events in which the collapse of Δψm constitutes an early, obligatory, and irreversible step of the apoptotic process. ¹⁸FTPP accumulates mainly in the mitochondria, and in direct correlation with Δψₘ. Cells affected by the treatment will accumulate ¹⁸FTPP much less than non-affected cells. Therefore, a significant change between pre- and post-treatment scan will indicate tumors responding to treatment, and lack of differences will indicate non-responding tumors. Collapse of Δψₘ occurs within hours after treatment with most therapeutic agents.

The ability to monitor the first event of the irreversible phase of the apoptotic process affords a noninvasive method for early and sensitive detection of tumor response to treatment. In the clinical setting, the imaging methods provided by the present disclosure offer a powerful tool for tailoring chemotherapy strategies to provide the most benefit to a patient, while reducing morbidity. The radiolabeled imaging compounds of the present disclosure are also suitable for use in developing these new generations of chemotherapy agents. For instance, the ¹⁸FTPP compounds of the present disclosure and imaging methods of using the same are suitable for use as a non-invasive technique for an early and sensitive assessment at the molecular level of treatment efficacy in clinical studies. Moreover, the imaging methods of the present disclosure are suitable for use in selecting suitable malignant targets in test subjects, based on the functional integrity of mitochondria, upon which a novel drug can be tested.

The present disclosure further provides imaging methods suitable for use in the imaging of tumors with imaging compounds of the present disclosure, particularly compounds including ¹⁸FTPP. In preferred tumor imaging methods of the present disclosure, the ¹⁸FTPP imaging compound administered to a patient preferentially accumulates in mitochondria of malignant cells such that the concentration of ¹⁸FTPP is greater in the mitochondria of the malignant cell than the concentration of the cation in adjacent normal cells.

The extent of cancerous disease (stage) is a major prognostic factor, and non-invasive staging using imaging technologies has a key role in design of treatment strategies (e.g., surgery vs. radio-chemotherapy vs. adjuvant chemotherapy). The ¹⁸FTPP compounds of the present disclosure accumulate in malignant cells to a substantially greater extent than in normal cells. Thus, administration of an imaging compound including ¹⁸FTPP is suitable for the identification and imaging of malignant cells and tumors and is further suitable for measuring the stage of tumor development.

The tumor imaging methods of the present disclosure are particularly suitable in certain embodiments for imaging of cancers, more particularly for imaging neoplasms including, but not limited to, a variety of lung, breast, and prostate cancers. Moreover, in embodiments, the tumor imaging methods of the present disclosure are capable of determining the extent of the cancerous disease (cancer stage).

The present disclosure further provides methods of imaging cardiovascular diseases, particularly methods of imaging the myocardia. The cardiovascular imaging methods of the present disclosure comprise the administration of at least one compound including ¹⁸FTPP to a patient suffering from or susceptible to a cardiovascular disease.

[¹⁸F]phosphonium cations (e.g., ¹⁸FTPP) of the present disclosure are suitable for various cardiovascular diseases, particularly myocardial imaging. Myocytes contain the highest concentration of mitochondria, and therefore the heart is by far the major organ target of phosphonium cations. In addition, phosphonium cations maintain excellent perfusion characteristics permitting high-contrast imaging of the heart. Infarct and heart failure involve apoptosis followed by necrosis processes. [¹⁸F]Phosphonium cations are capable of accurately distinguishing between myocardial segments in which the apoptotic process cannot be stopped by medication and revascularization, and myocardial areas that can be salvaged by intervention.

Preferred imaging methods provided by the present disclosure include the use of ¹⁸FTPP compounds and/or salts capable of generating at least a 2:1 target to background ratio of radiation intensity, or more preferably about a 5:1, about a 10:1 or about a 15:1 ratio of radiation intensity between target and background. In certain preferred methods, the radiation intensity of the target tissue is more intense than that of the background. In other embodiments, the present disclosure provides methods where the radiation intensity of the target tissue is less intense than that of the background. Generally, any difference in radiation intensity between the target tissue and the background that is sufficient to allow for identification and visualization of the target tissue is sufficient for use in the methods of the present disclosure.

In preferred methods of the present disclosure, the compounds of the present disclosure are excreted from tissues of the body quickly to prevent prolonged exposure to the radiation of the radiolabeled compound administered to the patient. Typically compounds of the present disclosure, including ¹⁸FTPP and salts thereof, are eliminated from the body in less than about 24 hours. More preferably, compounds of the present disclosure are eliminated from the body in less than about 16 hours, 12 hours, 8 hours, 6 hours, 4 hours, 2 hours, 90 minutes, or 60 minutes. Typically, preferred compounds are eliminated in between about 60 minutes and 120 minutes.

Preferred compounds of the present disclosure are stable in vivo such that substantially all, e.g., more than about 50%, 60%, 70%, 80%, or more preferably 90% of the injected compound is not metabolized by the body prior to excretion. Compounds and salts of the present disclosure and imaging methods of the present disclosure are useful in imaging a variety of conditions including cancer, cardiovascular and liver diseases, HIV, AIDS, autoimmune disease, degenerative disorders, neoplasms, and the like.

Typical subjects to which compounds of the present disclosure may be administered will be mammals, particularly primates, especially humans. For veterinary applications, a wide variety of subjects will be suitable, e.g. livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine or tissue samples, or blood urine or tissue samples of the animals mentioned for veterinary applications.

The present disclosure also provides packaged pharmaceutical compositions comprising a pharmaceutically acceptable carrier and a compound of the disclosure or salt thereof comprising at least one pharmaceutically acceptable anion and a radiolabeled cation according to the disclosure (e.g., ¹⁸FTPP). In certain embodiments, the packaged pharmaceutical composition includes the reaction precursors to be used to generate the compound or salt according to the present disclosure upon combination with a radiolabeled precursor. Other packaged pharmaceutical compositions provided by the present disclosure further include indicia including at least one of: instructions for using the composition to image cells or tissues having increased or suppressed mitochondrial activity, instructions for using the composition to assess therapeutic effect of a drug protocol administered to a patient, instructions for using the composition to selectively image malignant cells and tumors in the presence of inflammation, and instructions for using the composition to measure mitochondrial membrane potential (Δψₘ).

In certain preferred embodiments, the present disclosure provides a kit including from about 1 to 30 mCi of the radionuclide-labeled imaging agent described above (preferably ¹⁸FTPP) in combination with a pharmaceutically acceptable carrier. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like.

The present disclosure further provides apparatuses and synthetic protocols for the automated synthesis of ¹¹C, ¹⁸F, ⁷⁶Br, ¹²³I, ¹²⁴I or ¹³¹I labeled compounds of the present disclosure, including ¹⁸FTPP, and preparation of pharmaceutical compositions comprising same. The half-life (120 min) of ¹⁸F allows for distribution of cationic probes from a central cyclotron to satellite PET scanners. Tagging the cationic probes with I-123 allows for distribution from a manufacturing center to medical institutions equipped with SPECT.

Preferred embodiments of the disclosure include novel methods of synthesizing ¹⁸FTPP. In an embodiment ¹⁸FTPP is synthesized in a two-step process via direct coupling of triphenylphosphine (PPh3) with 4-[¹⁸F]Fluoroidodobenzene ([¹⁸F]FIB). In an embodiment, the two-step process includes the following steps: (1) synthesis of no-carrier added [¹⁸F]FIB using 1-trimethylamino-4-iodobenzene as a precursor; and (2) directly coupling the [¹⁸F]FIB produced in step 1 with PPh₃ to form ¹⁸FTPP. This process is described in greater detail in Example 1, below. In another embodiment ¹⁸FTPP can be synthesized in a one-step process involving direct nucleophilic substitution of no-carrier-added [¹⁸F]fluoride with the precursor 4-nitrophenyltriphenylphosphonium, which is described in greater detail in Example 2, below.

Imaging agents of the present disclosure may be used in accordance with the methods of the present disclosure by one of skill in the art (e.g., radiologists and other specialists in nuclear medicine) to image sites having a dysfunctional mitochondria (e.g., mitochondria exhibiting aberrant activity) in a sample, subject and/or patient. Any site having a dysfunctional mitochondria (e.g., mitochondria exhibiting aberrant activity) may be imaged by the imaging methods and imaging agents of the present disclosure.

Images can be generated by virtue of differences in the spatial distribution of the imaging agents that accumulate at a site having a dysfunctional mitochondria, e.g., mitochondria exhibiting aberrant activity. The spatial distribution may be measured using any imaging apparatus suitable for the particular label, for example, a gamma camera, a PET apparatus, a SPECT apparatus, and the like. The extent of accumulation of the imaging agent may be quantified using known methods for quantifying radioactive emissions. A particularly useful imaging approach employs more than one imaging agent to perform simultaneous studies. Alternatively, the imaging method may be carried out a plurality of times with increasing administered dose of the pharmaceutically acceptable imaging composition of the present disclosure to perform successive studies using the split-dose image subtraction method, as are known to those of skill in the art.

Preferably, a detectably effective amount of the imaging agent of the present disclosure is administered to a subject. In accordance with the present disclosure, “a detectably effective amount” of the imaging agent of the present disclosure is defined as an amount sufficient to yield an acceptable image using equipment that is available for clinical use. A detectably effective amount of the imaging agent of the present disclosure may be administered in more than one injection. The detectably effective amount of the imaging agent of the present disclosure can vary according to factors such as the degree of susceptibility of the individual, the age, sex, and weight of the individual, idiosyncratic responses of the individual, the dosimetry, and the like. Detectably effective amounts of the imaging agent of the present disclosure can also vary according to instrument and film-related factors. Optimization of such factors is well within the level of skill in the art.

The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

### EXAMPLES

Example 1

Materials and Methods

No-carrier-added [¹⁸F]Fluoride was produced at the Molecular Imaging Program, PETtrace cyclotron (General Electric Health Care, Waukesha, Wis.) by irradiation of enriched [¹⁸ O]water via the ¹⁸O(p,n)¹⁸F nuclear reaction. All other reagents were purchased from Sigma-Aldrich Chemical Co. ESI-MS were performed by Vincent Coates Foundation Mass Spectrometry Laboratory, Stanford University. HPLC was performed on a Dionex Summit® HPLC system (Dionex Corporation, Sunnyvale, Calif.) equipped with a 170U 4-Channel UV-Vis absorbance detector and radioactive detector (Carroll & Ramsey Associates, model 105S, Berkeley, Calif.). UV detection wavelengths are 225 nm and 280 nm for all the experiments. Both semi-preparative (GRACE Vydac C18, 9.4 mm×250 mm, CAT# 218TP510) and analytical (Dionex Acclaim®120 C18, 4.6 mm×250 mm) RP-HPLC columns were used. The mobile phase was solvent A, water/0.1% trifluoroacetic acid (TFA), and solvent B, acetonitrile/0.1% TFA. CRC-15R PET dose calibrator (Capintec Inc., Ramsey, N.J.) was used for all radioactivity measurements.

Cell Culture and Cell Uptake Studies of Phosphonium Cations

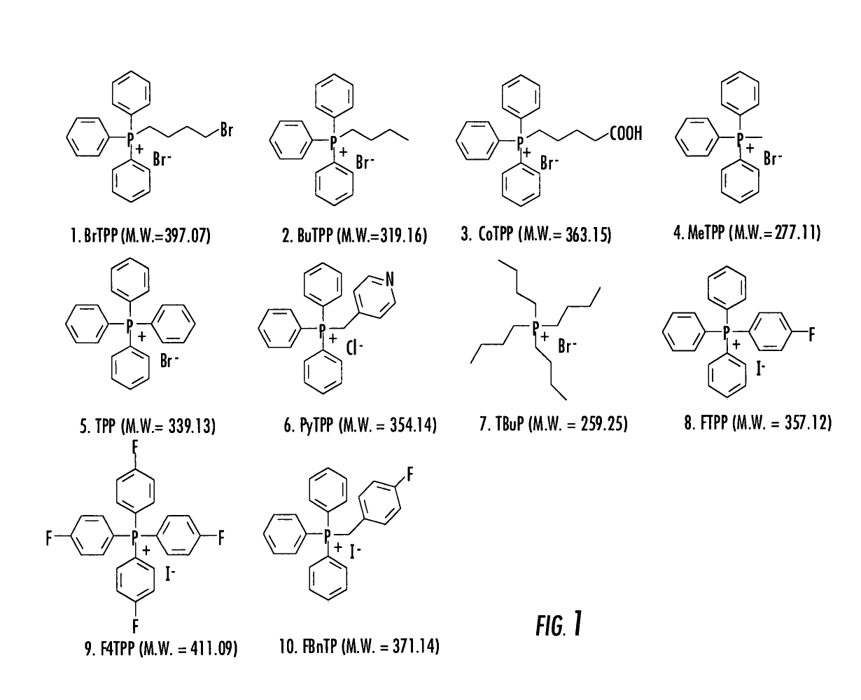

A rat glioma C6 cell line was grown in Dulbecco's modified Eagle medium (DMEM, Invitrogen Life Technologies, Carlsbad, Calif.) high glucose (containing KCl 5.3 mmol/L and NaCl 110.34 mmol/L) plus 10% fetal bovine serum (FBS), and 1% penicillin-streptomycin at 37° C. in 5% CO₂/95% air. After trypsinization, cells were washed with 0.01 M phosphate-buffered saline (PBS, pH=7.4) twice and suspended at 10×10⁶ cells /mL in low K⁺ Hepes buffer (135 mM NaCl/5 mM KCl/1.8 mM CaCl₂/0.8 mM MgSO₄/50 mM Hepes [4-(-2-hydroxyethyl)-1-piperazineethanesulfonic acid]/5.5 mM dextrose, pH 7.4). 50 μL (0.5×10⁶) of C6 cells were incubated with 10 μL 100 μM stock phosphonium salts solution (5 M final concentration, the structures of these phosphonium cations are shown in FIG. 1) and 140 μL Hepes buffer from 10 to 90 min at room temperature. The cells were centrifuged (250×g) for three minutes and washed twice with cold PBS buffer at specified times. Cell pellets were then lysed with 150 μL cold spectral grade water, following by freezing in dry ice. Right before the MALDI-TOF-MS analysis, the frozen cell solution was thawed and centrifuged at 12000×g for 5 min. 90 μL cell lysate and 10 μL 10 μM internal standard MeTPP were mixed together, and 1 μL solution was subjected to MALDI-TOF-MS analysis. Samples were prepared and analyzed in triplicate.

Mitochondrial Membrane Potential (MMP) Dependent Uptake of FTPP

The extent of MMP and cell membrane potential-dependent cellular uptake of FTPP was further assessed using the C6 cells treated with CCCP, valinomycin, and high K⁺ Hepes buffer (5 mM NaCl/135 mM KCl/1.8 mM CaCl₂/0.8 mM MgSO₄/50 mM Hepes/5.5 mM dextrose, pH 7.4). Carbonylcyanide-m-chlorophenylhydrazone (CCCP), a known protonphore that selectively abolishes the MMP,²⁶ was dissolved in dimethyl sulfoxide (DMSO) and diluted to desired concentration with low K⁺ Hepes buffer. K⁺-ionophore valinomycin was dissolved in ethanol and also diluted with low K⁺ buffer. Final concentrations of ethanol and DMSO were below 0.1%. Varying concentrations of CCCP and valinomycin were added to the 0.5×10⁶ cells in low K⁺ Hepes buffer 30 min prior to the start of the experiment. After 1 hr of incubation with 5 μM FTPP, the eppendorf vials were centrifuged, cell lysate were prepared as described above, and the cell uptakes of FTPP were determined using MALDI-TOF-MS. Five sets of 0.5×10⁶ cells were also suspended in high K⁺ Hepes buffer for 60 min, then exposed to 5 μM FTPP for 10-90 min in high K⁺ Hepes buffer. Cell uptake of FTPP was measured by MALDI-TOF-MS. The viability and the integrity of the cells after treatment with inhibitors and high K⁺ buffer were examined using Trypan blue. It showed that the cells were not affected by the inhibitors and high K⁺ buffer used within the time period when uptake of the phosphonium analogs was measured.

Radiosynthesis and HPLC purification of (4-[¹⁸F]Fluorophenyl)triphenyl-phosphonium cation

[¹⁸F]Fluoride was added to a pyrex glass reaction vessel containing 200 ml 25 mM potassium carbonate and Kryptofix 2.2.2. (3.0-4.0 mg) dissolved in 300 ml CH₃CN. The solution was evaporated at 120° C. by bubbling nitrogen gas, and the residue was dried by azeotropic distillation with acetonitrile (3×0.5 ml). To this anhydrous residue was added a solution of 1-trimethylamino-4-iodobenzene (6-10 mg) in dry DMAA (1.2 ml). The reaction mixture was heated for 5 min at 140° C. in a heating block. The solution was cooled down and passed a silica cartridge. DMM (2 mL) was then used to rinse the 4-[¹⁸F]Fluoroiodobenzene ([¹⁸F]FIB) from the silica cartridge into a glass vial.

Triphenylphosphine (PPh₃, 10 mg) dissolved in 100 μL DMM and 2.5 mg of palladium acetate were added into a small glass reaction vial, and 1 mL [¹⁸F]FIB collected from the above preparation was then added. The reaction mixture was heated for 60 min at 150° C. in an oil bath. The solution was cooled, diluted with 10 mL water, and passed through a C-18 cartridge. CH₃CN (2 mL) was then used to rinse out the crude radiolabeled product from the C-18 cartridge. The solution was injected onto a semipreparative HPLC column (the flow rate was 3 ml/min, with the mobile phase starting from 28% solvent B (CH₃CN/0.1% TFA) and 72% solvent A (H₂O/0.1% TFA) (0-3 min) to 38% solvent B and 62% solvent A at 33 min, then going to 85% solvent B and 15% solvent A (33-36 min), maintaining this solvent composition for another 3 min (36-39 min), and returning to initial solvent composition by 42 min). Pure [¹⁸F]FTPP, eluted out the column with a retention time of 24.4 min, was collected in a small round bottle. The product was dried in a rotary evaporator and was made isotonic with sodium chloride and passed through a 0.22 mm membrane filter into a sterile multidose vial. The product was found to be >95% radiochemically and chemically pure, as determined by analytical HPLC (same gradient as used for semi-preparative HPLC; flow rate: 1.0 ml/min). The retention time for [¹⁸F]FTPP under analytical HPLC conditions was 12.9 min. Radiochemical yield was usually around 0.5% (EOS; corrected for decay).

MicroPET Imaging

PET imaging of normal nude mice was performed on a microPET R4 rodent model scanner (Concorde Microsystems Inc, Knoxville, Tenn.). The mice were injected with about 10-20 μCi of [¹⁸F]FTPP via the tail vein. At 30 min, 1 hr, 2 hr, and 4 hr post injection, the mice were anesthetized with 2% isoflurane and placed in the prone position and near the center of the filed of view of microPET. The 10-min static scans were obtained, and the images were reconstructed by a two-dimensional ordered subsets expectation maximum (OSEM) algorithm. Regions of interest (ROIs) were then drawn over the tumor on decay-corrected whole-body coronal images. The counts per pixel per minute were obtained from the ROI and converted to counts per milliliter per minute by using a calibration constant. By assuming a tissue density of 1 g/mL, the ROIs were converted to counts/g/min. An image ROI-derived percentage ID per gram of tissue (% ID/g) was then determined by dividing counts per gram per minute with injected dose (ID).

Statistical Method

Statistical analysis was performed using the Student's t-test for unpaired data. A 95% confidence level was chosen to determine the significance between groups, with P<0.05 being significantly different.

Results and Discussion

Cell Uptake of Phosphonium Cations

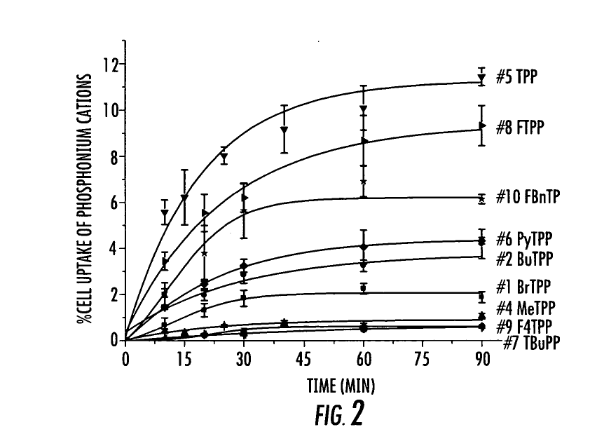

The C6 glioma cell uptake of a small library of phosphonium cations (FIG. 1) was evaluated first in this study. FIG. 2 shows the cell uptake of phosphonium cations (5 μM was used for incubation for all experiments) on C6 cells at 25° C. over a 90 min incubation period. The phosphonium cations exhibited very similar cell uptake kinetics. The cations rapidly increased in cells upon introduction into the buffer and reached steady state concentration at 60 min. The percentage of cell uptake for various phosphonium cations displayed different levels of steady-state ranging from 0.6% to 11.4%. Interestingly, TPP happens to be the one with the highest uptake in the C6 cells. FTPP exhibited 80% of the TPP uptake in C6 cells in vitro, which is ranked as 2^{nd} place in the compounds tested and has a much higher accumulation than that of 4-fluorobenzyltriarylphosphonium, methyltriphenylphosphonium, and butyltriphenyl-phosphonium.

MMP Dependent Uptake of FTPP

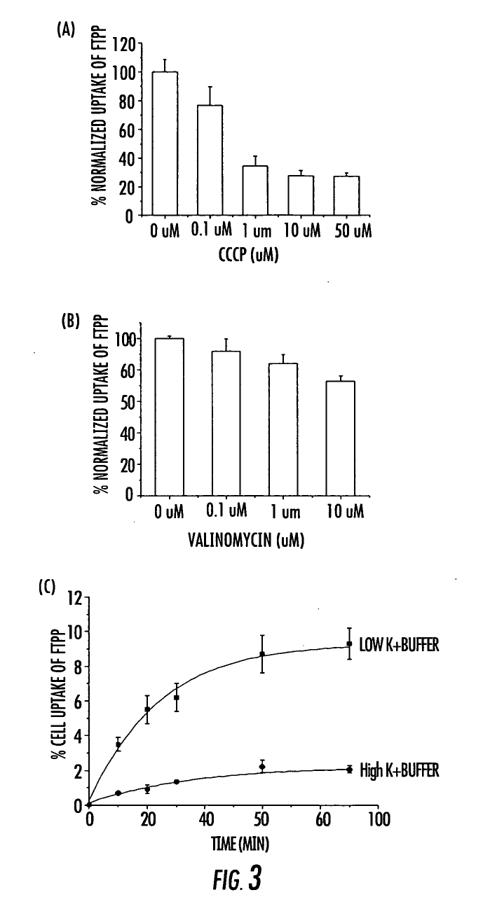

In order to assess the effects of manipulating mitochondrial membrane potential on the cellular accumulation of FTPP (determined by MALDI-TOF-MS), uptake studies were performed for C6 cells under 4 different concentrations of CCCP (0.1, 1, 10, 50 μM) and 3 different concentrations of valinomycin (0.1, 1, 10 μM) in low K⁺ Hepes buffer. For control experiments in which the mitochondrial membrane potentials were unaltered, uptake was determined in a near physiological buffer (low K⁺ Hepes buffer) without adding any inhibitors. The results are depicted in FIGS. 3A and 3B. It can be seen that the uptake of FTPP in the C6 cells is inhibited by CCCP or valinomycin in dose-dependent manners. The FTPP uptake is highly inhibited by 50 μM CCCP to about 27.3±2.3%, and to a lesser extent by 10 μM valinomycin to about 72.8±3.6% (FIG. 3).

The effects of depolarizing the plasma membrane potential only on the C6 cell uptake of the phosphonium cations were obtained by using a high K⁺ Hepes, as shown in FIG. 3C. For C6 rat glioma cells, depolarization of the plasma membrane caused a 3.5-fold decrease in uptake of FTPP, compared to their uptakes in the low K⁺ buffer at 90 min exposure time. Overall, these results clearly demonstrate that the uptake of FTPP is electrogenic and driven by the plasma and mitochondrial membrane potentials.

Radiosynthesis of [¹⁸]FTPP

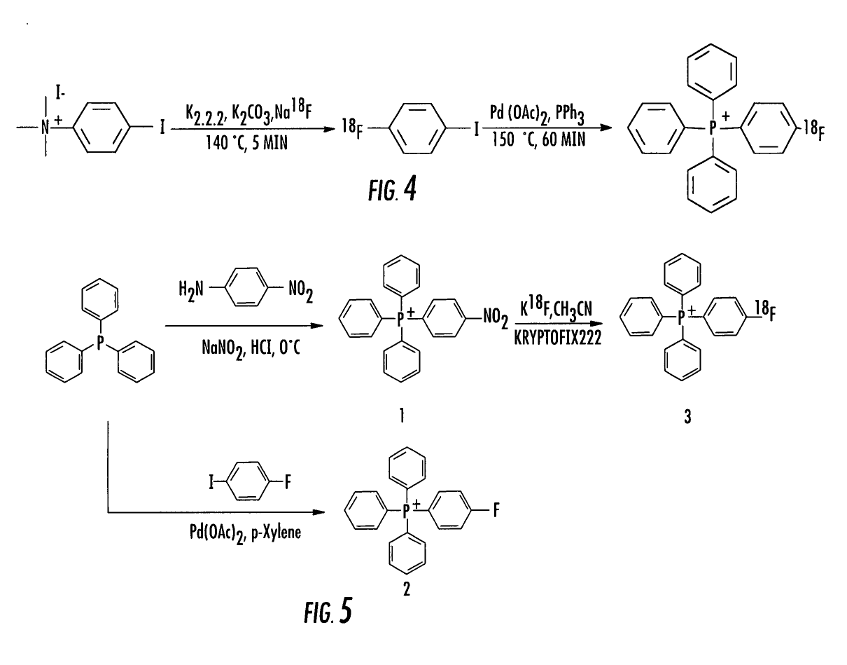

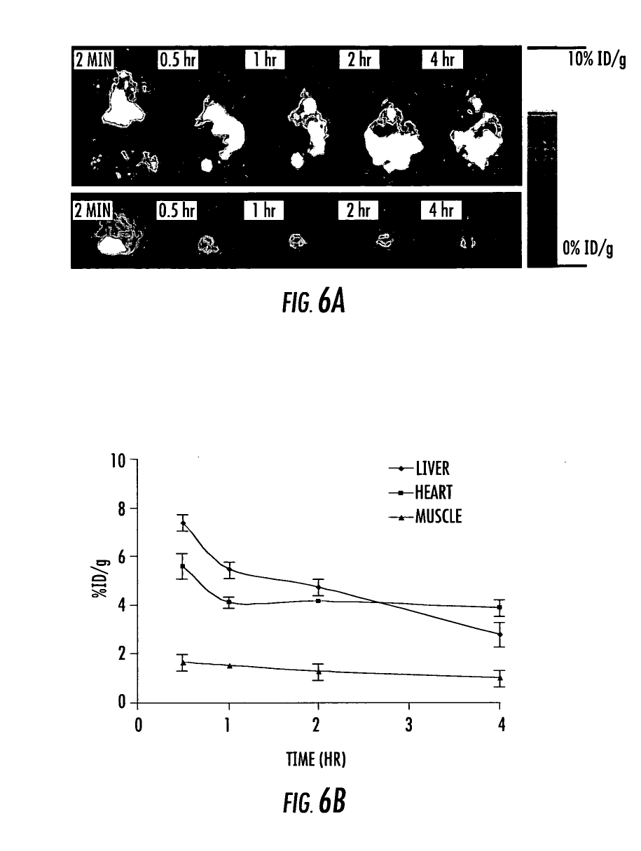

The radiosynthesis of ¹⁸FTPP via a two step procedure is shown in FIG. 4. Another method for rasiosynthesis of ¹⁸FTPP is illustrated in FIG. 5 and described in detail in Example 2, below. For the present synthesis procedure, no-carrier added [¹⁸F]FIB was first synthesized by using the procedure reported by Gail, R, Coenen, H H, Appl Radiat Isot 1994; 45: 105-111 (which is hereby incorporated by reference herein in its entirety), with minor modification. The 1-trimethylamino-4-iodobenzene was used as a precursor and subjected to nucleophilic exchange reaction with fluoride-18 in DMAA. Moderate radioachemical yield (around 5-10%) for producing [¹⁸F]FIB was usually achieved in 15 minutes. [¹⁸F]FIB was then used directly to couple with PPh₃ to form C-P bond under Pd(OAc)₂ catalysis. After heating for 1 hour at 150° C. in the mineral oil bath, the reaction solution was cooled and injected onto the semi-preparative HPLC column to separate the labeled product. Under the HPLC gradient described in the experimental section, ¹⁸FTPP with retention time 24.4 min was collected. Analytical HPLC analysis revealed that the radiolabeled product exhibited identical retention time (12.9 min) with a fully characterized cold ¹⁹FTPP standard. The radiochemical purity of ¹⁸FTPP, as determined by analytical HPLC, was above 95%, and the radiochemical yield for ¹⁸FTPP was around 0.5% at end of synthesis (EOS).

MicroPET Imaging of [¹⁸F]FTPP

The biodistribution and pharmacokinetic of [¹⁸F]FTPP in normal nude mice was performed by multiple time-point static microPET scans. FIG. 6A shows coronal and transverse microPET images of a female nude mouse at different times pi of 15 μCi of the radiotracer. All micro-PET images were decay corrected. The [¹⁸F]FTPP clearly localized in the heart, which is consistent with high mitochondria density in cardiac tissue. Moreover, some activity was also observed to localize in liver and intestines, indicating that the tracer was mainly cleared out through hepatobiliary system. Quantification analysis of images, illustrated in FIG. 6B, shows that the heart uptake of [¹⁸F]FTPP is 5.59±0.93% ID/g at 30 minutes pi. It is slowly washed out from the heart, even at 4 h pi, 3.89±0.29% ID/g heart uptake of the probe was still observed. However, the probe shows faster clearance in liver than that of in the heart (7.39±0.36 at 30 minutes pi and 2.79±0.59 4 h pi). These data also suggest that [¹⁸F]FTPP may be useful for imaging heart disease such as ischemia.

In conclusion, radiosynthesis of 4-[¹⁸F]Fluorophenyl)triphenylphosphonium in high radiochemical purity was achieved through two-steps reaction, and microPET imaging studies demonstrated that the probe has a great potential for imaging mitochondrial dysfunction.

### Example 2

The radiosynthesis of (4-[¹⁸F]fluorophenyl)triphenylphosphonium (¹⁸FTPP) via a one-step procedure is shown in FIG. 5 and described in greater detail below. The precursor for radiosynthesis, 4-nitrophenyltriphenylphosphonium (1, NO₂TPP) was first prepared by following the procedure reported by Rieke et al. (Rieke R D, White C K, Milliren C M. J Am Chem Soc 1976; 98: 6872-6877, which is hereby incorporated by reference herein in its entirety). The diazonium chloride intermediate was formed by slowly adding hydrochloric acid to 4-nitroaniline at 0° C. The formation of NO₂TPP was obtained by the addition of triphenylphosphine. Extraction with aqueous solution and precipitation with sodium iodide (Nal) afforded pure precursor NO₂TPP which was confirmed by analytical HPLC, ESI-MS and NMR. ¹⁸FTPP was synthesized by nucleophilic substitution reaction of NO₂TPP and no-carrier-added [¹⁸F]fluoride in the presence of potassium carbonate (K₂CO₃) and phase transfer agent Kryptofix 222. After heating for 15 min at 120° C. in the mineral oil bath, the reaction solution was cooled and injected onto the semipreparative HPLC column to separate the labeled product. These steps are discussed in additional detail below and illustrated in FIG. 5.

Experimental

No-carrier-added [¹⁸F]Fluoride was obtained from PETNET Pharmaceuticals, Inc. (Palo Alto, Calif.). All other reagents were purchased from Sigma-Aldrich Chemical Co. ESI-MS were performed by Vincent Coates Foundation Mass Spectrometry Laboratory, Stanford University. NMR spectra were recorded on a Varian 600 MHz instrument. Melting points were determined on an Electrothermal's TA 9100 digital melting point instrument (Barnstead/Thermolyne, Dubuque, Iowa). HPLC was performed on a Dionex Summit1 HPLC system (Dionex Corporation, Sunnyvale, Calif.) equipped with a 170U 4-Channel UV-Vis absorbance detector and radioactive detector (Carroll & Ramsey Associates, model 105S, Berkeley, Calif.). UV detection wavelengths are 225 and 280 nm for all the experiments. Both semi-preparative (Zorbax SB-C18, 9.4 mm×250 mm) and analytical (Dionex Acclaim1120 C18, 4.6 mm×250 mm) RP-HPLC columns were used. The mobile phase was solvent A, 100 mM ammonium formate (HCO₂NH₄), and solvent B, acetonitrile. CRC-15R PET dose calibrator (Capintec Inc., Ramsey, N.J.) was used for all radioactivity measurements.

Synthesis of 4-nitrophenyltriphenylphosphonium iodide

NO₂TPP was prepared as a precursor for radiosynthesis of ¹⁸FTPP by the method reported previously by Rieke et al. Briefly, 2.8 g (0.02 mol) 4-nitroaniline was mixed with 10 ml concentrated hydrochloric acid at 0° C. Then NaNO₂ (0.02 mol, 1.38 g) dissolved in cold 10 ml H₂O was added to the 4-nitroaniline dropwise with stirring, followed by adding 5.6 g sodium acetate (NaOAc) dissolved in water (20 ml). Triphenylphosphine (5.6 g) dissolved in 80 ml ethyl acetate was then added dropwise to the reaction vial at 0° C. within 60 min.

After incubation at room temperature for 5 min, the reaction solution was acidified by adding concentrated HCl (2 ml). The aqueous layer was then washed twice using ether, and the ethyl acetate layer was extracted twice with water. The aqueous solutions were then combined, and Nal (2.2 g in 2 ml H₂O) was added to triturate the final product. The purity of 4-nitrophenyltriphenylphosphonium iodide was over 95% as confirmed by analytic HPLC (analytic column, flow rate: 1 ml/min, elution protocol starts with 95% A for 3 min, followed by a linear gradient to 15% A over 30 min, retention time: 23.4 min). The yield was 6.34 g (62%), pale brown solid; m.p. >200° C. (lit25 2288C).

From the HMBC (multiple-bond CH correlation, 600 MHz, CDCl3) experiment, two aromatic hydrogens (8.57-8.58 ppm) were observed coupled with C-2 and C-6 (127 ppm) carbons as doublet, and two other aromatic hydrogens (8.09-8.10 ppm) were observed coupled with C-5, C-7 (137 ppm) as doublet of doublet with integral values 1:1 accordingly (the carbon connected to NO₂-group is numbered as 1, and the other carbons in the same benzene ring are numbered clockwise as 2-6, the same numbering system for carbon is applied for FTPP). This indicates that the NO₂-group is in para position relative to phosphorus atom. The rest of protons are combined in 7.6-8.1 ppm interval with integral value of 15 protons, thus it support the total integral ratio of 19 protons for compound NO₂TPP. ESI-MS (high-resolution): m/z calculated: 384.1153 (C₂₄H₁₉NO₂P), found: 384.1153.

Synthesis of (4-fluorophenyl)triphenylphosphonium formate

¹⁹FTPP was synthesized according to the method reported for synthesis of (4-bromophenyl) triphenylphosphonium (Lambert C, Gaschler W, Noll G, Webber M, Schmalzlin E, Brauchle C, Meerholz Klaus. J Chem Soc Perkin Trans 2 2001; 6: 964-974, which is hereby incorporated by reference herein in its entirety). Triphenylphosphine (1.76 g, 6.72 mmol), 4-fluoro-iodobenzene (1.49 g, 6.72 mmol) and Pd(OAC)₂ (75 mg, 0.33 mmol, 5 mol %) in p-xylene (40 ml) were mixed together and stirred at 130-140° C. for 2 h. The color changed from yellow to brown and a white precipitate formed. The solvent was removed in vacuo. 100 mg residue was dissolved in CH₃OH and purified by HPLC using semi-preparative column (flow rate: 3 ml/min; elution protocol starts with 95% of A for 3 min, followed by a linear gradient to 15% of A over 30 min, retention time: 23.8 min). The purity of the product was analyzed by analytical HPLC (analytic column, flow rate: 1 ml/min, same gradient as that for NO₂TPP analysis, retention time: 24.1 min, >95% purity). (White solid, m.p. >200° C.; three experiments.) 1H NMR, HMBC and HSQC (heteronuclear single quantum correlation) (600 MHz, CDCl3) were also performed for confirmation of the structure of FTPP. From these experiments, two protons (7.42-7.54, multiplet) coupled with C-2 and C-6 (118 ppm) carbons and two other aromatic hydrogens (7.68-7.75 ppm, multiplet) coupled with C-3 and C-5 (138 ppm) were observed, which demonstrates that the fluorine-group is in para position relative to phosphorus atom. The rest of protons are in the range of 7.58-7.67 ppm (multiplet, six protons), 7.76-7.84 (multiplet, six protons), and 7.85-7.96 ppm (triplet, three protons), supporting the total integral ratio of 19 protons for compound 2. ESI-MS (high-resolution): m/z calculated: 357.1208 (C24H19FP), found: 357.1217.

Radiosynthesis and HPLC purification of (4-[¹⁸F]fluorophenyl) triphenylphosphonium cation

[¹⁸F]fluoride was added to a pyrex glass reaction vessel containing 200 ml 25 mM potassium carbonate and Kryptofix 2.2.2. (3.0-4.0 mg) dissolved in 300 ml CH₃CN. The solution was evaporated at 120° C. by bubbling nitrogen gas and the residue was dried by azeotropic distillation with acetonitrile (3×0.5 ml). To this anhydrous residue was added a solution of NO₂TPP (1-1.5 mg) in dry DMSO (0.32 ml). The reaction mixture was heated for 15 min at 120° C. in an oil bath. The solution was cooled and injected onto a semipreparative HPLC column (the flow rate was 3 ml/min, with the mobile phase starting from 28% solvent B (CH₃CN) and 72% solvent A (0.1M HCO₂NH₄ in water) (0-3 min) to 48% solvent B and 52% solvent A at 33 min, then going to 85% solvent B and 15% solvent A (33-36 min), maintaining this solvent composition for another 3 min (36-39 min) and returning to initial solvent composition by 42 min). Pure [¹⁸F]FTPP, eluted off the column with a retention time of 26 min, was collected in a small round bottle. The product was dried in a rotary evaporator and was made isotonic with sodium chloride and passed through a 0.22 mm membrane filter into a sterile multidose vial. The product was found to be >95% radiochemically and chemically pure as determined by analytical HPLC (same gradient as used for semi-preparative HPLC; flow rate: 1.0 ml/min). The retention time for [¹⁸F]FTPP under analytical HPLC conditions was 25.5 min. Radiochemical yield ranged between 10 and 15% (EOS; corrected for decay). Thus starting typically with 100 mCi of [¹⁸F]-fluoride ion, about 7 mCi of product, ready for injection, was routinely obtained in 60 min.

Results and discussion

Under the HPLC gradient described above, the retention time of ¹⁸FTPP and un-reacted NO₂TPP were 26.0 and 24.5 min, respectively, which made the separation of these two species feasible. Analytical HPLC analysis revealed that the radiolabeled product exhibited identical retention time with a fully characterized cold (4-[¹⁹F]fluorophenyl)triphenylphosphonium (FTPP) standard. The radiochemical purity of ¹⁸FTPP determined by analytical HPLC was above 95%, and radiochemical yield for ¹⁸FTPP was 10-15% at end of synthesis (EOS) with specific activity 576-715 Ci/mmol.

It should be emphasized that the above-described embodiments of the present disclosure are merely possible examples of implementations and/or merely set forth for a clear understanding of the principles of the disclosure. Many variations and modifications may be made to the above-described embodiment(s) of the disclosure without departing substantially from the principles of the disclosure. All such modifications and variations are intended to be included herein within the scope of the present disclosure and following claims.

We claim:

1. A pharmaceutically acceptable imaging composition comprising:
(4-[¹⁸F]fluorophenyl)triphenylphosphonium (¹⁸FTTP), or a pharmaceutically acceptable salt thereof; and
a pharmaceutically acceptable carrier.

2. The imaging composition of claim 1, wherein the pharmaceutically acceptable salt comprises a ¹⁸FTTP cation and a pharmaceutically acceptable anion.

3. The imaging composition of claim 1, wherein the pharmaceutically acceptable anion is selected from I⁻, Cl⁻, and Br⁻.

4. An imaging probe comprising (4-[¹⁸F]fluorophenyl)triphenylphosphonium (¹⁸FTTP), or a pharmaceutically acceptable salt thereof.

5. The imaging probe of claim 4, wherein the probe is taken up by mitochondria and wherein the uptake is related to a change in a mitochondiral membrane potential (Δψₘ).

6. An imaging probe comprising a radiolabeled (4-[X]phenyl) triphenylphosphonium analog, wherein X comprises a radioisotope and wherein X is selected from: ¹¹C, ¹⁸F, ⁷⁶Br, ¹²³I, ¹²⁴I, and ¹³¹I.

7. The imaging probe of claim 6, wherein the probe is taken up by mitochondria and wherein the uptake is related to a change in a mitochondiral membrane potential (Δψₘ).

8. A method of imaging comprising:
providing an imaging probe comprising (4-[¹⁸F]fluorophenyl)triphenylphosphonium (¹⁸FTTP), or a pharmaceutically acceptable salt thereof
contacting a specimen to be imaged with a detectably effective amount of the imaging probe; and
making a radiographic image.

9. The method of claim 8, wherein the specimen is selected from: a cell, tissue, and a host.

10. The method of claim 9, wherein the host is a mammal.

11. The method of claim 9, wherein the tissue comprises cardiovascular tissue.

12. The method of claim 8, wherein making the radiographic image comprises using an imaging apparatus and wherein the imaging apparatus is selected from: a gamma camera, a PET apparatus, and a SPECT apparatus.

13. The method of claim 8, wherein the imaging comprises imaging changes in a mitochondiral membrane potential (Δψₘ).

14. The method of claim 8, wherein the imaging probe is taken up by mitochondria and wherein the uptake is related to a change in a mitochondiral membrane potential (Δψₘ).

15. A method of imaging a condition associated with mitochondrial dysfunction in a host comprising:
administering a detectably effective amount of a composition comprising (4-[¹⁸F]fluorophenyl)triphenylphosphonium (¹⁸FTPP) or a pharmacutically acceptable salt thereof to a host; and
creating a radiographic image of the location and distribution of the ¹⁸FTPP in the host with an imaging apparatus,
wherein the ¹⁸FTPP is taken up by mitochondria and wherein the uptake is related to a change in a mitochondiral membrane potential (Δψₘ).

16. The method of claim 15, wherein the mitochondrial dysfunction is selected from: increased activity and decreased activity.

17. The method of claim 15, wherein the mitochondrial dysfunction is indicated by a change in themitochondiral membrane potential (Δψₘ).

18. The method of claim 15, wherein the condition associated with mitochondrial dysfunction is selected from: cancer, diabetes, heart failure, cardiovascular diseases, liver diseases, HIV, AIDS, degenerative diseases, autoimmune disorders, myopathies, and conditions associated with aging.

19. The method of claim 15, wherein the imaging apparatus is selected from: a gamma camera, a PET apparatus, and a SPECT apparatus.

20. The method of claim 15, wherein imaging a condition associated with mitochondrial dysfunction comprises diagnosing the condition or monitoring the condition.

21. The method of claim 15, wherein the pharmaceutically acceptable salt comprises a ¹⁸FTTP cation and a pharmaceutically acceptable anion.

22. The method of claim 15, wherein the pharmaceutically acceptable anion is selected from I⁻, Cl⁻, and Br⁻.

23. A method of determining an effect of a drug comprising:
administering an amount of the drug to a host;
administering a detectably effective amount of a composition comprising (4-[¹⁸F]fluorophenyl)triphenylphosphonium (¹⁸FTPP) or a pharmacutically acceptable salt thereof to a host;
creating a radiographic image of the location and distribution of the ¹⁸FTPP in the host with an imaging apparatus,
determining an amount of ¹⁸FTPP taken up by host mitochondria,
wherein the amount of uptake by host mitochondria is related to the effect of the drug on apoptosis in host cells.

24. The method of claim 23, wherein the drug increases apoptosis.

25. The method of claim 23, wherein the drug decreases apoptosis.

26. A method of synthesizing (4-[¹⁸F]fluorophenyl)triphenylphosphonium (¹⁸FTPP) comprising the steps of:
syntheising no-carrier added [¹⁸F]FIB using 1-trimethylamino-4-iodobenzene as a precursor; and
coupling the [¹⁸F]FIB with PPh₃ to form ¹⁸FTPP.

27. A method of synthesizing (4-[¹⁸F]fluorophenyl)triphenylphosphonium (¹⁸FTPP) comprising the steps of:
providing no-carrier-added [¹⁸F]fluoride and 4-nitrophenyltriphenylphosphonium
performing direct nucleophilic substitution of no-carrier-added [¹⁸F]fluoride with the 4-nitrophenyltriphenylphosphonium to form ¹⁸FTPP.
